(19) United States
(12) Patent Application Publication
(10) Pub. No.: US 2010/0218460 A1
(43) Pub. Date: Sep. 2, 2010

(54) PROCESS AND INSTALLATION FOR THE CONTINUOUS VACUUM PREPACKING OF FOOD PRODUCTS

(75) Inventors: Suzanne LE GOFF-LARROCHE, Pujols (FR); Brigitte LARROCHE, Paris (FR)

Correspondence Address: YOUNG & THOMPSON, 209 Madison Street, Suite 500, Alexandria, VA, 22314, US

(73) Assignee: SODETECH SARL, La Teste de Buch (FR)

(21) Appl. No.: 12/685,031

(22) Filed: Jan. 11, 2010

(30) Foreign Application Priority Data
Mar. 2, 2009 (FR) 09/00966

(51) Int. Cl. B65B 55/02; B65B 31/04; B65B 63/08

(52) U.S. Cl. 53/425; 53/432; 53/440; 53/86

(57) ABSTRACT

Process for the continuous vacuum prepacking of food products that can be pasteurized and/or sterilized in a container closed by a cover that has no hooking element and is equipped with an elastic seal maintained by a forced vacuum, and the cover is opened by a vacuum-breaking system. During the placing under vacuum, the cover is kept apart from the previously filled container, superheated vapor is injected at a temperature, a flow rate and/or a time that allow the sterilization of the cover and the head space of the container by expelling air and incondensable products, in particular oxygen; the container is then pressed against the cover or vice versa, and a jet of cold water is sent onto the cover, which causes rapid cooling of the cover, the immediate condensation of the vapor phase in the head space of the container that allows the placing under deep vacuum.

This invention relates to a process and an installation for the continuous vacuum prepacking of food products that can be pasteurized or sterilized in a container made of glass, metal or plastic that is closed by a cover that has no hooking means and is equipped with an elastic seal that is maintained by a forced vacuum and whereby said cover is opened by a vacuum-breaking system.

French patent REY No. 77,09415 describes a process for the prepacking and preservation of perishable materials and the packaging for its use.

This patent describes a process in which vapor is injected between the cover and the container, and then the container is inserted into an autoclave, creating an overpressure in the latter, this overpressure being maintained for the entire duration of pasteurization or sterilization, and then the container is gradually cooled and the normal pressure is reestablished.

The overpressure inside the autoclave is mentioned as an essential means of the invention that is obtained by injection of compressed air in the autoclave for keeping the capsule or cover on the container.

Actually, it seems that it is impossible to pasteurize or to sterilize by injecting or introducing compressed air and vapor at the same time into an autoclave; the mixture of air and vapor is heterogeneous, and the temperature in the autoclave is thereby also heterogeneous. Only the special autoclaves that are equipped with enormous fans inside the autoclave can use this mixture by making it homogeneous by very strong mixing of the inside fan of the autoclave.

Likewise, the sterilization time that is necessary for obtaining the safety of preservation necessarily leads to overbaking of the product. It is unthinkable to allow the product to cool in the autoclave without intervening by rapid cooling that is carried out by a cold water circuit in the autoclave. The very slow cooling that the text recommends would bring about a degradation of any product by a caramelization of sugars or a degradation of tastes and color by the Maillard effect or the two associated effects.

The patent CATRAIN No. 92,00372 resorts to the same processes and means as the REY patent and is subject to the same impossibilities and drawbacks.

The invention relates to a process and an installation that makes it possible to carry out continuously, and without use of an autoclave being necessary, a vacuum prepacking of food products that can be pasteurized or sterilized in a rigid glass or metal container that is closed by a cover that has no means of hooking with the container and is equipped with an elastic seal, whereby the cover is maintained by a forced vacuum and whereby said cover is opened by a vacuum-breaking system, which avoids the drawbacks of the prior art.

In particular, the purpose of the invention is to avoid the overbaking of the product, its caramelization, and to not modify the taste and the color of the product.

For this purpose, the invention is essentially characterized in that the cover is kept at a distance from the previously emptied container, the superheated vapor is injected at a temperature, a flow rate and in a period that allows the decontamination of the cover and the head space of the container by expelling both the air and the incondensable products, in particular oxygen, which produces the vacuum, the container is pressed against the cover or vice versa, and a stream of cold water is sent onto the cover, which causes rapid cooling of the cover, the immediate condensation of the vapor phase in the head space of the container, and the placing under deep vacuum.

Advantageously, the cover is kept at a distance, 5 to 10 millimeters, from the previously filled container. The vapor that is injected between the cover and the container is superheated at 120°-135° to have a decontaminating effect and also to reduce the volume of condensate in the packaging.

Other characteristics and advantages of the invention will emerge upon reading the description below of the process and the installation for implementation illustrated by way of nonlimiting example by the accompanying drawings, in which:

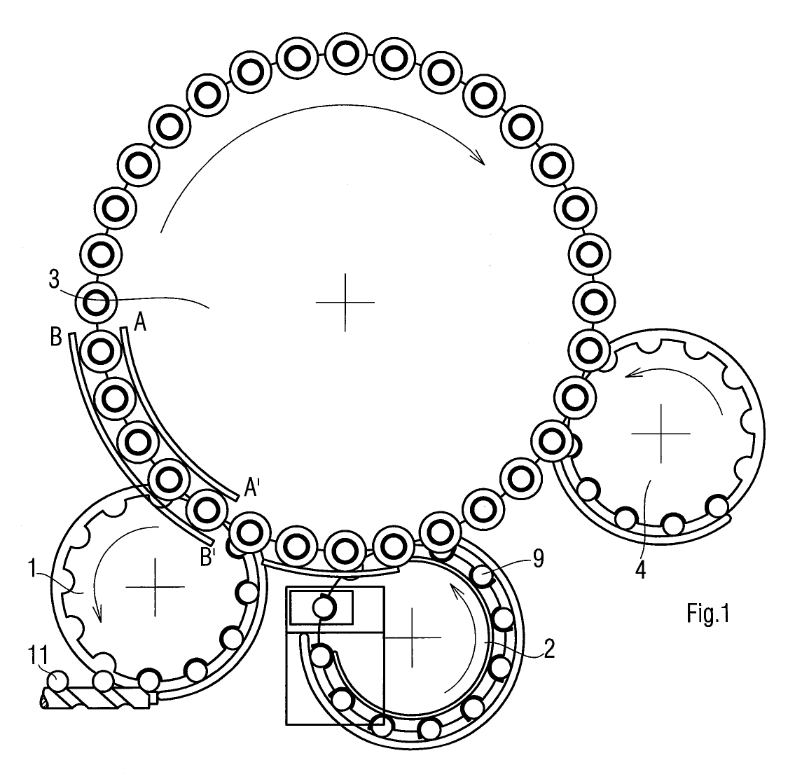

FIG. 1 is a schematic view of the installation that implements the process according to the invention;

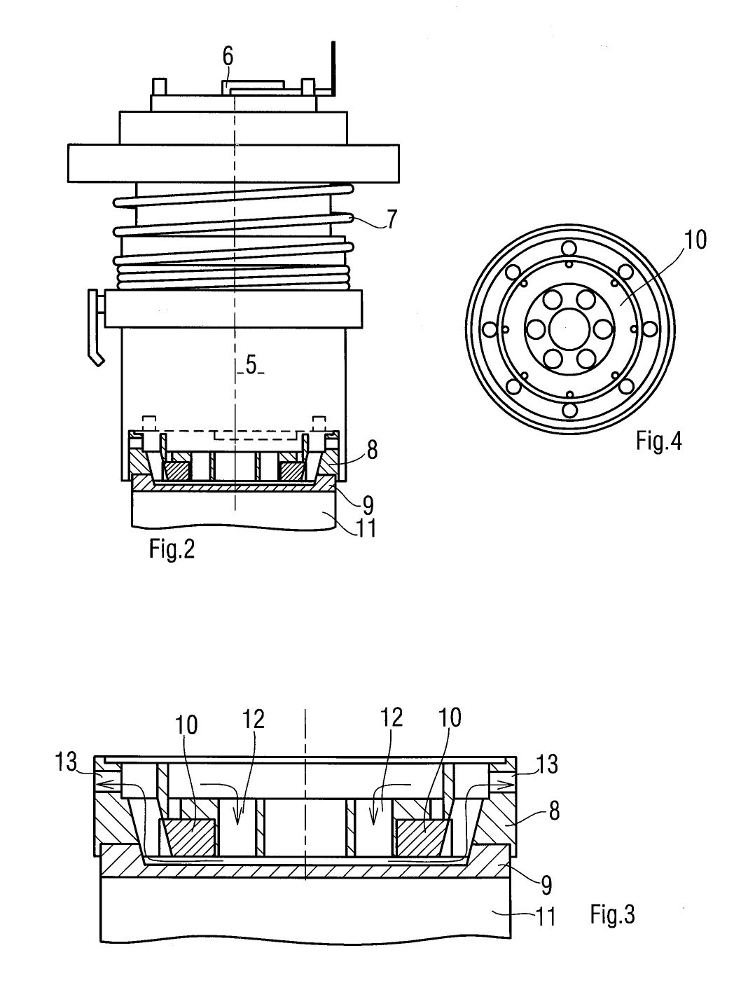

FIG. 2 is a cutaway view of the device for installation of the covers on the container;

FIG. 3 is a cutaway view of the moving mandrel for installation of the cover that is organized for carrying out rapid cooling according to the invention;

FIG. 4 is a bottom view of the mandrel with cover-holding magnets;

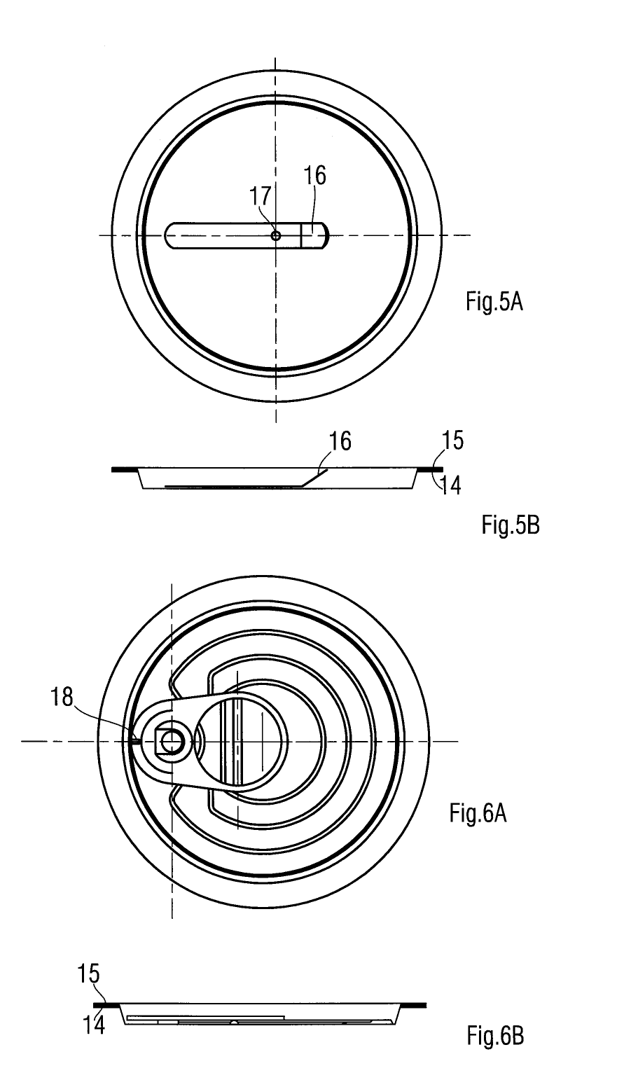

FIGS. 5A-5B show a cover with vacuum-breaking by taking off a heat-sealed tab;

FIGS. 6A-6B illustrate a cover with vacuum-breaking of the percussion ring type;

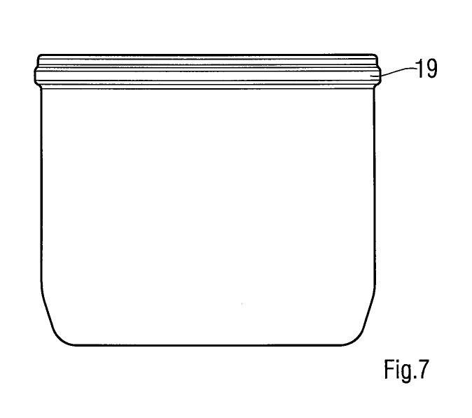

FIG. 7 shows a type of container.

The invention relates to a process and its means of implementation (unit, installation, etc., . . . ) for the continuous vacuum prepacking of food products, before or after pasteurization or sterilization, in a rigid container that is closed with a rigid cover, locked into position by the pressure differential between the inside of the container and the outside.

The rigid cover does not have any means for mechanical hooking to the container; the latter is easily opened by a vacuum-breaking system.

The process according to the invention can also be used for prepacking a super-clean product that is designed for cold-chain distribution without pasteurization or sterilization.

The packages or containers that are used in the implementation of the process should meet the following specifications as much as possible:
Either glass jars whose edges are optionally rounded and optionally equipped with grooves in the thickness to allow easy adhesion of the cover seal;
Or metal boxes whose edge is rolled or folded, creating a support and a surface to which the cover seal will adhere;
Or jars made of a synthetic material called plastic provided that they have a structure that offers good resistance to the pressure difference between the outside and the inside of the container.

In all of the cases, it is preferable that the containers, jars or box, comprise a keeper ring in peripheral relief under the level of the cover:
So as to prevent the adjacent cover edges from touching during transfers on the line after closing;
So that the keeper ring in relief creates an attachment zone for any over-cover that can be made of any suitable materials, plastic material, metals, cardboard, which thus makes possible a partial consumption of products and the blocking of the container. The keeper ring also has a function of rigidifying the neck of the box in the case of a metal or plastic box.

The metal cover that is used has the following characteristics:
Having a composition seal that is studied for being neither too rigid, nor too flexible, allowing a perfect adhesion to the container during the pressure between the outside and the inside of the container;
Preferably the cover is to have an unhooking or flap on the outside edge to interlock exactly with the flange of the container.

According to the invention, the process for the continuous vacuum prepacking of food products that can be pasteurized and/or sterilized (before or after closing) in a rigid container, of the type described above, that is closed by means of a metal cover that has no means or system for mechanical hooking to the container; whereby said cover is equipped with an elastic seal and maintained by a forced vacuum whose opening is made by a vacuum-breaking system. An injection of superheated vapor makes possible the decontamination of the cover and the head space of the container by expelling the air and the incondensable products, in particular oxygen, which produces the vacuum so that the container is pressed against the cover or vice versa, and so that a cold water jet is sent to the cover, which causes rapid cooling of the cover, the immediate condensation of the vapor phase in the head space of the container, and the placing under deep vacuum.

The product can be continuously sterilized in advance and then continuously cooled, whereby the product is encased at a temperature that is less than that of the pasteurized product in order to preserve the sugars and to prevent the caramelization and the proteins or the color and to prevent the Maillard reaction.

Advantageously, the containers as described move to the inlet of a filler in a preheating tunnel where they are subjected to a flow of superheated vapor to decontaminate their surface.

It is possible to provide a pre-sterilization of the container by means of a tunnel under ultraviolet rays or by a spraying of H₂O₂ that should precede the vapor flow so as to perfectly secure the decontamination.

This securing makes it possible to carry out an aseptic filling of the container, whereby the filling product can in this case be cold and sterile.

In the case of products that have to be pasteurized (products whose pH is less than 4.5), the containers are filled with the previously degassed hot product at a minimum temperature that makes possible a self-pasteurization of the container that is in contact with the product, for example at the temperature of 90° Centigrade by any filling means, for example a rotary metering device. In this case, the temperature of the product allows a self-pasteurization of the product itself and walls of the container that are in contact with the product.

The filled container is then transported toward a capping device.

The covers are placed in a tubular distribution storage unit and are subjected to decontamination by injection of superheated vapor (125° to 130°) optionally preceded by a spraying of H₂O₂.

They are then positioned respectively at a capping mandrel on which they are held by permanent magnets.

The containers are conducted over a substrate under each capping mandrel. They have previously circulated in a closed pipe or tunnel that contains superheated vapor such that the largest part of the incondensable gases can be eliminated from the head space of the container, which maintains the decontamination of the neck of the container.

The containers are positioned under the covers at a distance of 5 to 10 millimeters, and preferably 5 millimeters.

Superheated vapor injectors that inject said vapor into the space that is created between container and cover are arranged on each side of the container and the cover that is superposed at a distance of about 5 millimeters.

This superheated vapor injection carries out a flushing of the zone with the result of eliminating the residual air that is contained in the space between the cover and the product in the container, i.e., in the head space of the container.

The superheated vapor is applied at a temperature (120-135°), a flow rate and a period of time that makes it possible to complete the elimination of residual incondensable products from the head space of the container.

It was noted that the colder the product in the container was, the higher the flow rate of the vapor should be.

This final flushing has the effect of eliminating all of the incondensable gases, air and oxygen from the head space and of obtaining, after cooling, a deep vacuum, which prevents any degradation by oxidation on the product.

The mandrel that bears the cover is then supported and pressed onto the container or vice versa, which compresses the seal of the cover, and a jet of cold water is sent onto the cover by circulation of water in the mandrel, which causes rapid cooling, the immediate condensation of the vapor phase in the head space, and the placing under deep vacuum of said head space that keeps the cover in position.

The nature of the seal of the cover and the shape of the edges of the container make it possible to withstand the pressure differences and to ensure the hermeticism for the entire duration of marketing of the product.

After their closing and their partial cooling in the capsule-making device, the filled jars or boxes, now closed by a hermetic cover, then advance into a cooling tunnel.

Actually, the vacuum that is created in the jar or the box causes the boiling of the product, whereby the product is cooled by the heat that is taken by the boiling that creates the vapor. This vapor increases the pressure again in the head space, but it condenses immediately by contact with the cover on which the cooling water from the tunnel flows. The entire process takes place virtually at the same time and in a repetitive way, thus ensuring the very rapid cooling of the product.

The final vacuum is more intense than in the products that are prepacked in so-called “twist-off” jars, where a significant fraction of air remains, contrary to the process described here.

The jars or boxes pass through a drying tunnel and are ready for grouping and outer packaging.

A subsequent operation of sterilization in an autoclave can also take place in the event where the products require such a treatment for the purpose of a long preservation at ambient temperature. In this case, it will be ensured that the pressure outside of the container in the sterilization chamber is controlled so as to be always higher than the one that is naturally created by the heating of the product during sterilization inside the container during the temperature rise and fall phases.

The outside counter-pressure is to be at least 300 mbar higher than the pressure in the container. The pressure differential between the inside of the container and the chamber of the autoclave is not to exceed 950 mbar.

The product can be stacked in the container at low temperature, whereby the sterilization is carried out after the container is closed.

At the outlet of the rotary capsule-making unit, after the precooling that is carried out on the unit itself, the container has a pressure differential that depends on the temperature of the product that is contained; this differential is to be at least 300 mbar of vacuum so as to ensure that the cover is firmly held on the body during subsequent transfers.

The thus hermetically sealed container can pass into a sterilization apparatus (an autoclave) that is equipped with a so-called counter-pressure system.

The counter-pressure is to be at least 300 mbar higher than the pressure corresponding to the pressure that is produced in the container, by the temperature that is selected to obtain the desired sterilizing value (SV). The cooling of the container is to be performed in the autoclave that is accompanied by a counter-pressure that is to decrease in proportion as the product itself cools. The pressure differential between the inside of the container and the chamber of the autoclave is not to exceed 950 mbar.

This process also makes it possible to treat sterilized products in an aseptic method. In this case, the supply of the filler is to be preceded by a continuous sterilization of the product, followed by a cooling that is also continuous; the body of the filler is to be placed in an aseptic atmosphere zone, whereby the superheated vapor can be a simple and effective solution.

The product can be encased at a temperature that is less than the temperature of the pasteurized product. The temperature is provided for preserving the sugars (caramelization) or in the other case for preserving the proteins or the color (Maillard reaction). The product can even be encased completely under cold conditions.

At the outlet of the capsule-making unit for the pasteurization technique, the hermetically sealed jars or boxes are to pass into a cooling tunnel.

The advantages of the process are as follows:
Continuous manufacturing makes it possible to obtain higher-quality products economically,
The duration for preserving food products is improved by the absence of oxygen, by a reduced pasteurization scale, and by the rapid cooling that prevents any Maillard reaction and caramelization,
The food products can be preserved in glass, plastic or metal containers of any size,
The opening is free of any risk of cuts or metal chips falling in the product,
The opening is extremely easy even for the containers with large diameters, contrary to pre-packed products in so-called “twist-off” jars where it is necessary to limit the vacuum level so that the consumer can open the jar without too much difficulty,
The consumer has proof of the preservation and the integrity of the product by the noise that is created by activating the vacuum breaker, the click of the cover that expands, and the slight whistling of air that penetrates the container.

The accompanying drawings illustrate—diagrammatically and by way of nonlimiting example—an installation for continuous implementation of the invention.

FIG. 1 shows a top view of a carrousel 1 for supplying previously filled containers,

FIG. 2 shows the cover supply carrousel,

FIG. 3 shows a circular carrousel whose direction of rotation is indicated by the arrow and that passes successively to the following carrousels, in the vapor injection zone AA′-BB′ and on the carrousels 1 and 2 and then on the exit carrousel 4.

FIG. 2 shows a device for distributing covers of which a large number are carried by the carrousel 3.

This device comprises a hollow body 5 that is equipped at its top with a water supply valve 6, a compression spring 7, and at its base with a mandrel 8 that is equipped with permanent magnets 10 (FIG. 4).

The mandrel 8 that passes through the carrousel 2 takes the cover 9 and holds it by the action of the permanent magnet upon passing into the vapor injection zone AA′-BB′.

After the vapor injection, the container 11 is pushed toward the mandrel 8 or vice versa under the action of a piston, not shown.

This determines the pressurization of the cover 9 and of its flexible seal that is referenced as 14 in FIGS. 5 and 6 in pressure on the opening of the container 11. Jointly, this seal 14 is compressed on the edge of the container.

The mandrel 8 is supplied with cold water by the valve 6.

For this purpose, the mandrel is provided with at least one inlet opening of the water 12 up to the level of the cover 9 and at least one lateral outlet opening of the water 13, which carries out the cooling.

FIGS. 5A-5B show a cover with its seal 14, its flange or flap 15, and a vacuum-breaking device 16 that consists of a heat-sealed tab that blocks an opening 17 of the cover.

FIGS. 6A-6B show a cover that is equipped with a vacuum breaker 18 of the type that consists of a ring that strikes a determined zone by tilting and optionally is pre-cutout from the cover.

FIG. 7 shows a glass container that is equipped with its peripheral keeper ring 19 under its opening.

1. Process for the continuous vacuum prepacking of food products that can be pasteurized and/or sterilized in a container that is closed by a cover that has no means of hooking and is equipped with an elastic seal that is maintained by a forced vacuum [and the cover is opened by a vacuum-breaking system];
during the placing under vacuum, the cover (9) is kept apart from the previously filled container, superheated vapor is injected at a temperature, a flow rate and/or a time that allow the sterilization of the cover and the head space of the container by expelling the air and the incondensable products, in particular oxygen;
the container is then pressed against the cover or vice versa, characterized in that a jet of cold water is sent onto the cover, which causes rapid cooling of the cover, the immediate condensation of the vapor phase in the head space of the container that allows the placing under deep vacuum.

2. Process according to claim 1, wherein the cover (9) is kept at a distance, between 5 to 10 millimeters, from the previously filled container.

3. Process according to claim 1, wherein prior to the superheated vapor injection and to the placing of the cover, the containers before filling have undergone a flow of superheated vapor to heat them and to decontaminate their surfaces.

4. Process according to claim 2, wherein prior to the vapor injection and the placing of the cover (9), the containers before filling can be subjected to a presterilization by ultraviolet rays.

5. Process according to claim 2, wherein prior to the vapor injection and to the placing of the cover (9), the containers, before filling, can be subjected to a presterilization by spraying H₂O₂.

6. Process according to claim 1, wherein the vapor that is injected between the cover (9) and the head space of the container is superheated to 120°-135°.

7. Process according to claim 1, wherein the containers are filled with the hot product that was previously degassed at a minimum temperature allowing a self-pasteurization of the container in contact with the product.

8. Process according to claim 2, wherein the containers that are filled and closed can undergo a sterilization in an autoclave, whereby the pressure outside of the container is always higher than that inside the container during the temperature rise and fall phases.

9. Process according to claim 8, wherein the counter-pressure is to be at least 300 mbar higher than the pressure in the container.

10. Process according to claim 9, wherein the pressure differential between the inside of the container and the chamber of the autoclave is not to exceed 950 mbar.

11. Process according to claim 1, wherein the product is previously sterilized continuously and then cooled continuously, whereby the product is encased at a temperature that is lower than that of the pasteurized product in order to preserve the sugars and to prevent the caramelization and the proteins or the color and to prevent the Maillard reaction.

12. Installation for the implementation of the process according to claim 1 comprising:
A first container supply carrousel (1);
A second cover supply carrousel (2);
A third carrousel (3) that is equipped with mandrels (8) for gripping covers (9) using magnets (10) on the carrousel (2); characterized by:
A zone AA′ and BB′ of the carrousel (3) for vapor injection into the space that is created between the container and the cover;
A piston pushes the container (11) toward the mandrel (8) or vice versa;
A spring (7) is compressed, and a valve for supplying cooling water (10) is open, whereby this water is poured onto the cover (9), which causes the condensation of the vapor in the head space and the desired vacuum.

13. Installation according to claim 12, wherein each mandrel (8) is carried by a hollow body (5) on top of which is a compression spring (7) and a water supply valve (6) that opens by compression of the spring (7).

14. Installation according to claim 13, wherein the mandrel (8) is equipped with a water circuit that passes through the hollow body (5) on the cover (9) through at least one inlet (12) and at least one lateral outlet (13).
